(19) United States
(12) Patent Application Publication
(10) Pub. No.: US 2020/0124615 A1
(43) Pub. Date: Apr. 23, 2020

(54) METHODS FOR IDENTIFYING AND DE-EPITOPING ALLERGENIC POLYPEPTIDES

(71) Applicant: Ukko Inc., Mountain View, CA (US)

(72) Inventor: Yanay OFRAN, Tel-Aviv (IL)

(73) Assignee: Ukko Inc., Mountain View, CA (US)

(21) Appl. No.: 16/474,092

(22) PCT Filed: Dec. 28, 2017
(86) PCT No.: PCT/IB2017/058470

§ 371 Date: Jun. 27, 2019

Related U.S. Application Data
(60) Provisional application No. 62/440,309, filed on Dec. 29, 2016.

(51) Int. Cl. G01N 33/68; G16B 40/00; G16B 20/30; C12N 15/10; G01N 33/543

(52) U.S. Cl. CPC G01N 33/6845; G16B 40/00; C12N 15/1058; C12N 15/1037; G01N 33/54306; G16B 20/30

(57) ABSTRACT

Provided herein are methods for identifying one or more epitopes on allergenic polypeptides. Also provided herein are methods for de-epitoping allergenic polypeptides.

### FIELD OF THE INVENTION

The present disclosure relates to methods for identifying one or more epitopes on allergenic polypeptides, as well as to methods for de-epitoping allergenic polypeptides.

### BACKGROUND

Conservative estimates indicate that at least 3% of the general population suffers from food allergies, while some studies estimate the number as high as 30%. The economic impact of food allergies is substantial. Childhood food allergies alone were estimated to cost patients and their families $25 billion per year in the USA. While there are more than 170 types of foods known to cause allergic reactions, eight foods account for 90% of food allergy cases: peanuts, tree nuts, wheat, soy, milk, eggs, fish, and shellfish/crustaceans. For allergenic individuals, exposure to even microscopic amounts of an allergen may lead to life-threatening reactions.

Treatment of food allergies relies on strict avoidance of the trigger food(s). However, there are growing concerns over the nutritional impact of food avoidance. In particular, studies that evaluated growth measurements against diet records suggested that food allergies put children at risk for inadequate nutrition. Current treatment regimens for food allergies, including oral immunotherapy (OIT), have failed to cure patients, and safety concerns have been raised due to cases of both gastrointestinal and systemic reactions, sometimes requiring epinephrine.

Food allergies impair the quality of life of patients, their family, friends, and the society at large.

Background art includes WO 2016086185 and Sela-Culang et al., Structure (2014), 22, 1-12, 2014.

All references cited herein, including patent applications, patent publications, non-patent literature, and UniProtKB/Swiss-Prot Accession numbers are herein incorporated by reference in their entirety, as if each individual reference were specifically and individually indicated to be incorporated by reference.

### BRIEF SUMMARY

While current treatment of food allergies focuses on strict avoidance of the trigger foods, this can be difficult or impossible for allergic individuals, because even if allergic individuals diligently avoid knowingly consuming or coming into contact with an allergen, these individuals risk accidental exposure to trigger foods on a daily basis (e.g., in restaurants, airplanes and other forms of public transportation, workplaces, etc.). This can be particularly problematic for allergic school-aged children who are constantly exposed to potential trigger foods in the classrooms, lunchrooms, and cafeterias of their schools. New strategies to dealing with the economic, societal, and health impacts of food allergies other than food avoidance are urgently needed.

To meet the above and other needs, the present disclosure relates to a novel approach for coping with food allergies: modifying/mutating residues in the epitopes of allergen-specific antibodies on food allergens to reduce or eliminate the source of allergy in these foods for most/all patients suffering from allergy to the given food. Without wishing to be bound by theory, this novel approach to treating food allergens themselves (instead of, or in parallel to, treating the allergic individuals) is believed to provide a number of beneficial outcomes for allergic individuals, their families, and society at large: 1) providing foods and products that are safe to handle and consume for allergic individuals, 2) allowing allergic individuals to no longer avoid consuming otherwise healthy foods (thereby addressing the nutritional disadvantages of food avoidance), 3) reducing the risk of accidental exposure by increasing use of non-allergenic foods, 4) reducing the negative economic impacts of food allergies, 5) reducing/eliminating the need to perform potentially risky treatments on allergic individuals, 6) mapping epitopes for individual patients, thus offering the possibility of personalized tests that assess the risks of each person for various foods, 7) mapping epitopes for individual patients, thus offering the possibility of personalized nutrition recommendations, and 8) mapping epitopes for individual patients, thus offering the possibility of personalized treatment and exposure to environmental or medical reactants that are not necessarily food.

Accordingly, in one aspect, provided herein are methods for identifying an epitope on an allergenic polypeptide comprising the steps of: isolating serum and/or one or more allergen-specific B cells and/or one or more antibodies (e.g., IgE antibodies) from a subject; determining the sequence of one or more immunoglobulins from the serum and/or one or more allergen-specific B cells and/or one or more antibodies (e.g., IgE antibodies); identifying the antigen binding regions (ABRs) from the sequence of the one or more immunoglobulins; computationally predicting an epitope on an allergenic polypeptide for the identified ABRs; and validating the computationally predicted epitope experimentally, thereby identifying the epitope on the allergenic polypeptide.

In another aspect, provided herein are methods for de-epitoping an allergenic polypeptide comprising the steps of: isolating serum and/or one or more allergen-specific B cells and/or one or more antibodies (e.g., IgE antibodies) from a subject; determining the sequence of one or more immunoglobulins from the serum and/or one or more isolated allergen-specific B cells and/or one or more antibodies (e.g., IgE antibodies); identifying the antigen binding regions (ABRs) from the sequence of the one or more immunoglobulins; computationally predicting an epitope on an allergenic polypeptide for the identified ABRs; validating the computationally predicted epitope, thereby identifying the epitope on the allergenic polypeptide; and mutating one or more amino acid residues of the identified epitope on the allergenic polypeptide, thereby de-epitoping the allergenic polypeptide and abrogating its binding to the serum and/or allergen specific-B cell and/or one or more antibodies (e.g., IgE antibodies). In some embodiments, the method comprises mutating two or more, three or more, four or more, or five or more amino acid residues of the identified epitope. In some embodiments, the mutation is a conservative mutation.

In some embodiments, the mutation is a non-conservative mutation. In some embodiments, the mutation reduces allergenicity of the allergenic polypeptide relative to an allergenic polypeptide lacking the mutation. In some embodiments, the mutation eliminates allergenicity of the allergenic polypeptide. In some embodiments, the mutation reduces affinity of an antibody for the allergenic polypeptide relative to the affinity of the antibody to an allergenic polypeptide lacking the mutation. In some embodiments, the antibody is an IgA antibody, an IgE antibody, or an IgG antibody. In some embodiments, the antibody is an IgE antibody. In some embodiments, the affinity is reduced by at least about 10%, at least about 20%, at least about 30%, at least about 40% at least about 50%, at least about 75%, or at least about 99%. In some embodiments, the mutation reduces reactivity of serum isolated from the subject to the polypeptide relative to reactivity of the serum to a polypeptide lacking the mutation. In some embodiments, serum reactivity is reduced by at least about 10%, at least about 20%, at least about 30%, at least about 40% at least about 50%, at least about 75%, or at least about 99%. In some embodiments, the mutation eliminates reactivity of serum isolated from the subject to the polypeptide. In some embodiments, the mutation does not disrupt the function of the polypeptide. In some embodiments, the mutation does not disrupt the three-dimensional structure of the polypeptide. In some embodiments, the mutation does not disrupt folding of the polypeptide.

In some embodiments that may be combined with any of the preceding embodiments, the allergen-specific B cells are isolated form peripheral blood of the subject. In some embodiments that may be combined with any of the preceding embodiments, the subject suffers from a food allergy. In some embodiments, the food allergy is an allergy to peanuts, tree nuts, wheat, soy, milk, eggs, fish, shellfish, or any combination thereof. In some embodiments, the food allergy is a peanut allergy.

In some embodiments that may be combined with any of the preceding embodiments, the immunoglobulin is an IgA immunoglobulin, an IgE immunoglobulin, or an IgG immunoglobulin. In some embodiments, the immunoglobulin is an IgE immunoglobulin.

In some embodiments that may be combined with any of the preceding embodiments, the ABRs are identified using the online tool Paratome. In some embodiments that may be combined with any of the preceding embodiments, computationally predicting the epitope is performed using the online tool PEASE. In some embodiments that may be combined with any of the preceding embodiments, validating the computationally predicted epitope experimentally comprises performing an antibody cross blocking assay or an assay comprising screening a library. In some embodiments, the library is a yeast display library. In some embodiments, the library is a phage display library.

In another aspect, provided herein are methods for de-epitoping an allergenic polypeptide comprising the steps of: constructing a library comprising mutated polypeptides or polynucleotides encoding the mutated polypeptides, wherein the mutated polypeptides comprise one or more substitutions to the amino acid sequence of the allergenic polypeptide; assessing binding and/or reactivity of serum and/or one or more allergen-specific B cells and/or one or more antibodies isolated from a subject to the mutated polypeptides; and identifying mutated polypeptides with reduced binding and/or reactivity of serum and/or one or more allergen-specific B cells and/or one or more antibodies to the mutated polypeptides relative to the allergenic polypeptide. In some embodiments, binding and/or reactivity of the serum and/or one or more allergen-specific B cells and/or one or more antibodies to the mutated polypeptide is reduced by at least about 10%, at least about 20%, at least about 30%, at least about 40%, at least about 50%, at least about 75%, or at least about 99% relative to the allergenic polypeptide. In some embodiments, the allergen-specific B cells are isolated from peripheral blood of the subject. In some embodiments, the subject suffers from a food allergy. In some embodiments, the food allergy is an allergy to peanuts, tree nuts, wheat, soy, milk, eggs, fish, shellfish, or any combination thereof. In some embodiments, the food allergy is a peanut allergy. In some embodiments, the antibody is an IgA antibody, an IgE antibody, or an IgG antibody. In some embodiments, the antibody is an IgE antibody. In some embodiments, the library is a yeast display library. In some embodiments, the library is a phage display library. In some embodiments, the mutated polypeptides comprise two or more, three or more, four or more, or five or more substitutions to the amino acid sequence of the allergenic polypeptide. In some embodiments, the one or more substitutions are random substitutions. In some embodiments, the methods further comprise identifying one or more substitutions in the mutated polypeptides that reduce allergenicity of the allergenic polypeptide. In some embodiments, the one or more substitutions eliminate allergenicity of the allergenic polypeptide.

According to yet another aspect of the present invention there is provided a method for identifying an epitope on an allergenic polypeptide, the method comprising the steps of:

a) identifying antigen binding regions (ABRs) from the amino acid sequence of an antibody or T cell receptor which binds to the allergenic polypeptide; and
b) computationally predicting an epitope on the allergenic polypeptide for the identified ABRs, thereby identifying the epitope on the allergenic polypeptide.

According to yet another aspect of the present invention there is provided a method for de-epitoping an allergenic polypeptide, the method comprising mutating one or more amino acid residues of an epitope on the allergenic polypeptide to generate one or more mutation in the identified epitope, thereby de-epitoping the allergenic polypeptide.

According to embodiments of the present invention, the method further comprises:

a) identifying antigen binding regions (ABRs) from the amino acid sequence of an antibody or T cell receptor which binds to the allergenic polypeptide; and subsequently
b) computationally predicting an epitope on the allergenic polypeptide for the identified ABRs; wherein steps a) and b) are performed prior to the mutating.

According to embodiments of the present invention, the method further comprises validating the computationally predicted epitope experimentally.

According to embodiments of the present invention, the method further comprises validating the computationally predicted epitope experimentally following step b) and prior to the mutating.

According to embodiments of the present invention, the antibody or the T cell receptor is isolated from peripheral blood of an allergic subject.

According to embodiments of the present invention, the antibody is isolated from serum of an allergic subject.

According to embodiments of the present invention, the antibody or the T cell receptor is isolated from a subject that suffers from a food allergy.

According to embodiments of the present invention, the food allergy is an allergy to a food selected from the group consisting of legumes, tree nuts, sesame, wheat, soy, milk, eggs, fish, seafood and rice.

According to embodiments of the present invention, the food allergy is an allergy to a seed.

According to embodiments of the present invention, the food allergy is a peanut allergy.

According to embodiments of the present invention, the antibody is an IgA antibody, an IgE antibody, or an IgG antibody.

According to embodiments of the present invention, the antibody is an IgE antibody.

According to embodiments of the present invention, the ABRs are identified using the online tool Paratome.

According to embodiments of the present invention, the computationally predicting the epitope is performed using a machine-learning algorithm trained to recognize residue pairing preferences on a dataset of antibody/antigen complexes.

According to embodiments of the present invention, the computationally predicting the epitope is performed using a machine-learning algorithm trained to recognize whether a given antibody and a given antigen are likely to bind each other based on data set of antibody-antigen interactions.

According to embodiments of the present invention, the computationally predicting the epitope is performed using an algorithm trained to recognize HMC-peptide interaction for peptides derived from food proteins.

According to embodiments of the present invention, the computationally predicting the epitope is performed using an algorithm trained to predict TCR-MHC-peptide or TCR-peptide interaction for peptides derived from food proteins.

According to embodiments of the present invention, the antibody/antigen complexes comprise IgE antibody/antigen complexes.

According to embodiments of the present invention, the validating the computationally predicted epitope experimentally comprises performing an antibody cross blocking assay or an assay comprising screening a library.

According to embodiments of the present invention, the library is a yeast display library.

According to embodiments of the present invention, the library is a phage display library.

According to embodiments of the present invention, the mutating comprises mutating at least two amino acid residues of the identified epitope.

According to embodiments of the present invention, the mutation is a conservative mutation.

According to embodiments of the present invention, the mutation is a non-conservative mutation.

According to embodiments of the present invention, the mutation reduces allergenicity of the allergenic polypeptide relative to an allergenic polypeptide lacking the mutation, wherein the allergenicity is measured by binding of serum, skin prick test or clinical assessment of patients after exposure to the polypeptide.

According to embodiments of the present invention, the mutation eliminates allergenicity of the allergenic polypeptide in an allergic subject, wherein the allergenicity is measured by binding of serum, skin prick test or clinical assessment of patients after exposure to the polypeptide.

According to embodiments of the present invention, the mutation reduces the affinity of an antibody for the allergenic polypeptide relative to the affinity of the antibody to an allergenic polypeptide lacking the mutation, wherein the affinity is measured by a competition assay, ELISA, SPR or thermophoresis.

According to embodiments of the present invention, the antibody is selected from the group consisting of an IgA antibody, an IgE antibody, and an IgG antibody.

According to embodiments of the present invention, the antibody is an IgE antibody.

According to embodiments of the present invention, the affinity is reduced by at least about 10%.

According to embodiments of the present invention, the mutation reduces reactivity of serum isolated from the subject to the polypeptide relative to reactivity of the serum to a polypeptide lacking the mutation.

According to embodiments of the present invention, the serum reactivity is reduced by at least about 10%.

According to embodiments of the present invention, the mutation eliminates reactivity of serum isolated from the subject to the polypeptide.

According to embodiments of the present invention, the mutation does not disrupt the function of the polypeptide.

According to embodiments of the present invention, the mutation does not disrupt the three-dimensional structure of the polypeptide.

According to embodiments of the present invention, the mutation does not disrupt folding of the polypeptide.

According to yet another aspect of the present invention there is provided a method for de-epitoping an allergenic polypeptide, the method comprising the steps of:

a) constructing a library comprising mutated polypeptides or polynucleotides encoding the mutated allergenic polypeptides, wherein the mutated polypeptides comprise one or more modifications to the amino acid sequence of the allergenic polypeptide;
b) assessing binding of allergen-specific antibodies or allergen-specific T cell receptors to the mutated polypeptides; and
c) identifying mutated polypeptides with reduced binding to the allergen-specific antibodies or allergen-specific T cell receptors relative to the allergenic polypeptide.

According to embodiments of the present invention, the function of the mutated polypeptides is not disrupted.

According to embodiments of the present invention, the method further comprises assessing the expression, folding and/or function of the mutated polypeptides.

According to embodiments of the present invention, the binding is reduced by at least about 10% relative to the allergenic polypeptide.

According to embodiments of the present invention, the allergen-specific antibodies or the allergen-specific T cell receptors are isolated from peripheral blood of a subject.

According to embodiments of the present invention, the allergen-specific antibodies or the allergen specific T cell receptors are isolated from a subject that suffers from a food allergy.

According to embodiments of the present invention, the food allergy is an allergy to a food selected from the group consisting of legumes, tree nuts, sesame, wheat, soy, milk, eggs, fish, seafood and rice.

According to embodiments of the present invention, the food allergy is a peanut allergy.

According to embodiments of the present invention, the antibody is an IgA antibody, an IgE antibody, or an IgG antibody.

According to embodiments of the present invention, the antibody is an IgE antibody.

According to embodiments of the present invention, the library is a yeast display library.

According to embodiments of the present invention, the library is a phage display library.

According to embodiments of the present invention, the mutated polypeptides comprise at least two substitutions to the amino acid sequence of the allergenic polypeptide.

According to embodiments of the present invention, the one or more modifications are random modifications.

According to embodiments of the present invention, the method further comprises d) identifying one or more substitutions in the mutated polypeptides that reduce allergenicity of the allergenic polypeptide.

According to embodiments of the present invention, the one or more modifications eliminate allergenicity of the allergenic polypeptide.

According to yet another aspect of the present invention there is provided a method of inducing desensitization to a food in an allergic subject comprising providing a de-epitoped polypeptide of the food to the subject using a treatment regimen that induces desensitization to the food in the allergic subject, thereby inducing desensitization to food in the allergic subject. According to yet another aspect of the present invention there is provided a method of preventing allergenicity of a subject to a food comprising providing to the subject a de-epitoped polypeptide of the food to the subject, under conditions that prevents allergenicity of the subject to the food, thereby preventing allergenicity of the subject of the food.

According to embodiments of the present invention, the polypeptide is de-epitoped according to the method described herein.

It is to be understood that one, some, or all of the properties of the various embodiments described herein may be combined to form other embodiments of the present invention. These and other aspects of the invention will become apparent to one of skill in the art.

### BRIEF DESCRIPTION OF THE SEVERAL VIEWS OF THE DRAWINGS

FIGS. 1A-1B show the computationally predicted epitope residues (highlighted in red) on two allergenic polypeptides (shown in gold) that were solved in complex with their respective allergen-specific IgE antibodies (shown in grey). FIG. 1A shows the two strongest epitope residues on beta-lactoglobulin predicted for the complexed IgE antibody. FIG. 1B shows the two strongest epitope residues on the cockroach allergen Bla g 2 predicted for the complexed IgE antibody.

FIGS. 2A-2B show models of the structure and epitope(s) of the provicilin allergen.

FIG. 2A shows a model of the trimer organization of the provicilin allergen. FIG. 2B shows a model of one of the monomers of the provicilin allergen. Left and right snapshots represent two orientations of the provicilin allergen, rotated ˜90° relative to one another. Dark red, dark green, and dark blue patches represent the epitopes mapped for the light red, light green, and light blue monomers, respectively (as identified by the linear peptides).

FIG. 3 shows a schematic representing the protocol used for preparing libraries containing point mutations at selected positions generated by the dubbed incorporation synthetic oligos via gene reassembly (ISOR) method.

FIG. 4 shows a schematic representing the iterative stages for generating non-allergenic polypeptides.

### DETAILED DESCRIPTION

The present disclosure provides methods for identifying one or more epitopes on allergenic polypeptides, as well as methods for de-epitoping allergenic polypeptides. Advantageously, the methods described herein provide, for the first time, methods for identifying the epitopes on allergenic polypeptides (e.g., polypeptides from foods known or suspected of causing food allergies) of allergen-specific antibodies isolated from a subject, and methods of de-epitoping these allergenic polypeptides, thus providing polypeptides with reduced or eliminated allergenicity. Without wishing to be bound by theory, the methods described herein provide are believed to provide a number of beneficial outcomes for allergic individuals, their families, and society at large: 1) providing foods and products that are safe to handle and consume for allergic individuals, 2) allowing allergic individuals to no longer avoid consuming otherwise healthy foods (thereby addressing the nutritional disadvantages of food avoidance), 3) reducing the risk of accidental exposure by increasing use of non-allergenic foods, 4) reducing the negative economic impacts of food allergies, 5) reducing/eliminating the need to perform potentially risky treatments on allergic individuals, 6) mapping epitopes for individual patients, thus offering the possibility of personalized tests that assess the risks of each person for various foods, 7) mapping epitopes for individual patients, thus offering the possibility of personalized nutrition recommendations, and 8) mapping epitopes for individual patients, thus offering the possibility of personalized treatment and exposure to environmental or medical reactants that are not necessarily food.

Accordingly, in one aspect, provided herein are methods for identifying an epitope on an allergenic polypeptide comprising the steps of: isolating serum and/or one or more allergen-specific B cells and/or one or more antibodies (e.g., IgE antibodies) from a subject; determining the sequence of one or more immunoglobulins from the serum and/or one or more isolated allergen specific B cells and/or one or more antibodies (e.g., IgE antibodies); identifying the antigen binding regions (ABRs) from the sequence of the one or more immunoglobulins; computationally predicting an epitope on an allergenic polypeptide for the identified ABRs; and validating the computationally predicted epitope experimentally, thereby identifying the epitope on the allergenic polypeptide.

In another aspect, provided herein are methods for identifying an epitope on an allergenic polypeptide, the method comprising identifying antigen binding regions (ABRs) from the amino acid sequence of an antibody or T cell receptor which binds to the allergenic polypeptide; and computationally predicting residues from the allergenic polypeptide (i.e. residues of the epitope) to which the antibody or the T cell receptor binds, thereby identifying the epitope on the allergenic polypeptide.

In another aspect, provided herein are methods for de-epitoping an allergenic polypeptide comprising the steps of: isolating serum and/or one or more allergen-specific B cells and/or one or more antibodies (e.g., IgE antibodies) from a subject; determining the sequence of one or more immunoglobulins from the serum and/or one or more isolated allergen-specific B cells and/or one or more antibodies (e.g., IgE antibodies); identifying the antigen binding regions (ABRs) from the sequence of the one or more immunoglobulins; computationally predicting an epitope on an allergenic polypeptide for the identified ABRs; validating the computationally predicted epitope, thereby identifying the epitope on the allergenic polypeptide; and mutating one or more amino acid residues of the identified epitope on the allergenic polypeptide, thereby de-epitoping the allergenic polypeptide.

In another aspect, provided herein are methods for method for de-epitoping an allergenic polypeptide, the method comprising mutating one or more amino acid residues in an allergen, such that the expression, folding and function or the allergen are minimally affected but recognition by antibodies or T cell receptors or HMC molecules is hampered or abrogated, thereby de-epitoping the allergenic polypeptide.

In another aspect, provided herein are methods for de-epitoping an allergenic polypeptide comprising the steps of: constructing a library comprising mutated polypeptides or polynucleotides encoding the mutated polypeptides, wherein the mutated polypeptides comprise one or more substitutions to the amino acid sequence of the allergenic polypeptide; assessing binding and/or reactivity of serum and/or one or more allergen-specific B cells and/or one or more antibodies (e.g., IgE antibodies) isolated from a subject to the mutated polypeptides; and identifying mutated polypeptides with reduced binding and/or reactivity of serum and/or one or more allergen-specific B cells and/or one or more antibodies to the mutated polypeptides relative to the allergenic polypeptide.

In still another aspect there is provided a method for de-epitoping an allergenic polypeptide, the method comprising constructing a library comprising mutated polypeptides or polynucleotides encoding the mutated polypeptides, wherein the mutation does not disrupt (or minimally disrupts) the function of the allergenic polypeptide; assessing binding of allergen-specific antibodies or allergen-specific T cell receptors to the mutated polypeptides; and identifying mutated polypeptides with reduced binding to the allergen-specific antibodies or allergen-specific T cell receptors relative to the allergenic polypeptide.

The following description sets forth exemplary methods, parameters, and the like. It should be recognized, however, that such description is not intended as a limitation on the scope of the present disclosure but is instead provided as a description of exemplary embodiments.

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Gene Transfer Vectors for Mammalian Cells (J. M. Miller and M. P. Calos, eds., 1987); PCR: The Polymerase Chain Reaction, (Mullis et al., eds., 1994); Short Protocols in Molecular Biology (Wiley and Sons, 1999).

### Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used herein, the singular forms “a”, “an” and “the” include plural referents unless the content clearly dictates otherwise. Thus, for example, reference to “a molecule” optionally includes a combination of two or more such molecules, and the like.

As used herein, the term “about” refers to the usual error range for the respective value readily known to the skilled person in this technical field. Reference to “about” a value or parameter herein includes (and describes) embodiments that are directed to that value or parameter per se.

As used herein, the terms “polynucleotide”, “nucleic acid sequence”, “nucleic acid”, and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications.

As used herein, a nucleic acid is “operatively linked” or “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous.

As used herein, the term “vector” refers to discrete elements that are used to introduce heterologous nucleic acids into cells for either expression or replication thereof. An expression vector includes vectors capable of expressing nucleic acids that are operatively linked with regulatory sequences, such as promoter regions, that are capable of effecting expression of such nucleic acids. Thus, an expression vector may refer to a DNA or RNA construct, such as a plasmid, a phage, recombinant virus or other vector that, upon introduction into an appropriate host cell, results in expression of the nucleic acids. Appropriate expression vectors are well known to those of skill in the art and include those that are replicable in prokaryotic cells and/or eukaryotic cells, and those that remain episomal or those which integrate into the host cell genome.

As used herein, the terms “polypeptide,” “protein,” and “peptide” are used interchangeably and may refer to a polymer of two or more amino acids.

As used herein, the terms “mutated polypeptide”, “mutant polypeptide”, “de-epitoped polypeptide”, and “polypeptide comprising one or more mutations” refers to a polypeptide that has been genetically engineered to comprise one or more non-naturally occurring mutations.

As used herein, the term “protein allergen”, “allergenic polypeptide”, or “allergenic protein” refers to a polypeptide that is capable of inducing allergy or specific hypersensitivity in one or more individuals. Allergenic polypeptides may come from any organism.

As used herein, a “subject”, a “patient”, or an “individual” refers to any animal classified as a mammal, including, for example, humans, non-human primates, domestic and farm animals, and zoo, sports, or pet animals, such as dogs, horses, cats, cows, sheep, rabbits, as well as animals used in research, such as mice and rats, etc. In some embodiments, the mammal is human.

The term “allergen” refers to an antigen which elicits, induces, stimulates, or enhances an immune response by a cell (e.g. B cell or T cell) or the immune system of an exposed animal (e.g., human). An antigen is an allergen when the specific immune response is the development of enhanced sensitivity or a hypersensitivity to the antigen, but the antigen itself is not typically innately harmful. An allergen is therefore a particular type of antigen that can cause development of enhanced or increased sensitivity or hypersensitivity in a subject. For example, an allergen can elicit production of IgE antibodies in predisposed subjects.

The term “allergy” as used herein refers to a hypersensitivity disorder of the immune system (including a T-cell response).

The term “allergenicity” as used herein refers to the potential of an allergen to cause an allergic reaction.

An allergic response includes but is not limited to an allergic reaction, hypersensitivity, an inflammatory response or inflammation. In certain embodiments allergic response may involve one or more of cell infiltration, production of antibodies, production of cytokines, lymphokines, chemokines, interferons and interleukins, cell growth and maturation factors (e.g., differentiation factors), cell proliferation, cell differentiation, cell accumulation or migration (chemotaxis) and cell, tissue or organ damage or remodeling.

Allergic responses can occur systemically or locally in any region, organ, tissue, or cell. In particular aspects, an allergic response occurs in the skin, the upper respiratory tract, the lower respiratory tract, pancreas, thymus, kidney, liver, spleen, muscle, nervous system, skeletal joints, eye, mucosal tissue, gut or bowel.

The term “epitope” refers to any polypeptide determinant capable of specifically binding to an antibody or a T-cell receptor. In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. In certain embodiments an epitope may be a peptide or part thereof presented on MHC.

The term “antigen-binding site” refers to a portion of an antibody or T-cell receptor capable of specifically binding the epitope of an antigen. The antigen binding site may comprise at least one, two, three, four, five or all of the CDRs of the antibody. The antigen binding site may also in some embodiments be a paratope. In certain embodiments, an antigen-binding site is provided by one or more antibody or T cell receptor variable regions.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta chains. TCRs may exist in alpha/beta and gamma/delta forms, which are structurally similar but have distinct anatomical locations and functions. Each chain is composed of two extracellular domains, a variable and constant domain.

In some embodiments, the TCR may be modified on any cell comprising a TCR, including, for example, a helper T cell, a cytotoxic T cell, a memory T cell, regulatory T cell, natural killer T cell, and gamma delta T cell. TCRs in the present invention may exist in a variety of forms including different fragments of TCR with or without mutations.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations α and β light chains refer to the two major antibody light chain isotypes.

### Methods

As described above, the present disclosure relates, in part, to a novel approach to coping with food allergies by treating the allergens themselves instead of, or in parallel to, the allergic individuals. This novel approach includes methods of identifying allergen-specific antibody epitopes on known or predicted allergenic polypeptides, as well as methods of de-epitoping an allergenic polypeptide by modifying the identified epitopes, thereby reducing or eliminating allergenicity of the previously allergenic polypeptides.

Certain aspects of the present disclosure relate to a method for identifying an epitope on an allergenic polypeptide. In some embodiments, the method comprises isolating serum and/or one or more allergen-specific B cells and/or one or more antibodies from a subject;

determining the sequence of one or more immunoglobulins from the serum and/or one or more isolated allergen-specific B cells and/or one or more antibodies; identifying the antigen binding regions (ABRs) of the one or more immunoglobulins from the sequence of the one or more immunoglobulins; computationally predicting an epitope on an allergenic polypeptide for the identified ABRs; and validating the computationally predicted epitopes experimentally, thereby identifying the epitope on the allergenic polypeptide.

Other aspects of the present disclosure relate to a method for de-epitoping an allergenic polypeptide. In some embodiments, the method comprises identifying one or more epitopes on the allergenic polypeptide by any of the methods described herein, and mutating one or more amino acids residues of the one or more identified epitopes on the allergenic polypeptide. In some embodiments, the one or more mutations reduce or eliminate allergenicity of the polypeptide, thereby de-epitoping the previously allergenic polypeptide. In some embodiments, the one or more mutations reduce or eliminate allergenicity of the polypeptide relative to a polypeptide lacking the one or more mutations (e.g., a wild-type polypeptide). In some embodiments, the de-epitoped polypeptide is any of the polypeptides described herein.

Other aspects of the present disclosure relate to a method for de-epitoping an allergenic polypeptide through use of a library. In some embodiments, the method comprises constructing a library comprising mutated polypeptides or polynucleotides encoding the mutated polypeptides, wherein the mutated polypeptides comprise one or more substitutions to the amino acid sequence of any of the allergenic polypeptides described herein; assessing binding and/or reactivity of serum and/or one or more allergen-specific B cells and/or one or more antibodies (e.g., IgE antibodies) isolated from a subject to the mutated polypeptides by any of the methods as described herein; and identifying mutated polypeptides with reduced binding and/or reactivity of serum and/or one or more allergen-specific B cells and/or one or more antibodies to the mutated polypeptides relative to the allergenic polypeptide. Methods of making libraries comprising polypeptides comprising one or more substitutions, or libraries comprising polynucleotides encoding polypeptides comprising one or more substitutions are known in the art. In some embodiments, the library is a yeast display library. In some embodiments, the library is a phage display library. In some embodiments, the one or more substitutions are random substitutions. In some embodiments, the method further comprises identifying one or more substitutions in the mutated polypeptides that reduce allergenicity of the allergenic polypeptide. In some embodiments, the one or more substitutions eliminate allergenicity of the allergenic polypeptide.

Methods of isolating serum and antibodies from a subject are known in the art. Methods of isolating allergen-specific B cells from a subject are known in the art, including, for example, by the methods described in Patil, S. U. et al. (2015) J. Allergy Chu. Immunol. 136(1): 125-134. In some embodiments, the allergen-specific B cells are isolated from peripheral blood of the subject. Methods of isolating T cells and/or T cell receptors include cell sorting or sequencing of samples using TCR-specific primers. In some embodiments, the subject suffers from an allergy. In some embodiment, the allergy is a food allergy. In some embodiments, the food allergy is an allergy to peanuts, tree nuts, wheat, soy, milk, eggs, fish, shellfish, or any combination thereof. In some embodiments, the food allergy is a peanut allergy.

In one embodiment, the allergy is to seeds. As used herein, the term “seeds” refers to seeds of trees (i.e. nuts), seeds of pod-bearing plants (i.e. legumes) or seeds of fruit which comprise allergenic storage proteins. Examples of tree nuts include, but are not limited to walnut, pecan, almond, hazelnut, cashew, pistachio and Brazil nut. Other exemplary legumes include, but are not limited to alfalfa, clover, peas, beans, chickpeas, lentils, lupin bean, mesquite, carob, soybeans, peanuts and tamarind.

Other exemplary seeds contemplated by the present invention include, but are not limited to chestnuts, sesame, cocoa seeds, cotton seeds, flax seeds, macadamia nuts, mustard, pine nuts, poppy seeds, pumpkin seeds and sunflower seeds.

According to a particular embodiment, the seed is a peanut—e.g. of the species Arachis hypogaea.

Exemplary subspecies and varieties of Arachis hypogaea contemplated by the present invention include the subspecies fastigiata Waldron (exemplary varieties include, but are not limited to var. aequatoriana Krapov. & W. C. Greg; var. fastigiata (Waldron) Krapov. & W. C. Greg; var. peruviana Krapov. & W. C. Greg; and var. vulgaris Harz) and the subspecies hypogaea L. (exemplary varieties include hirsuta J. Kohler and var. hypogaea L).

In some embodiments, the antibody is isolated from the serum or from isolated B cells. In other embodiments, the antibody is isolated from peripheral blood. In other embodiments, the antibody is isolated from bone marrow. In other embodiments the T cell receptor is isolated from isolated T cells. Methods of sequencing antibodies and T cell receptors isolated from a subject are known in the art. Methods of amplifying and sequencing one or more portions of DNA from isolated cells are known in the art. In some embodiments, the immunoglobulins are IgA immunoglobulins, IgE immunoglobulins, IgG immunoglobulins, or any combinations thereof. In some embodiments, the immunoglobulins are IgE immunoglobulins. In some embodiments, the antigen binding regions (ABRs) of the one or more sequenced immunoglobulins are identified. In some embodiments, the ABRs are identified experimentally. Methods of identifying antigen binding regions on antibodies are known in the art, including, for example, by alanine scanning mutagenesis and crystallographic approaches. Methods of identifying ABRs are known in the art, including, for example, using antibody sequence or structural alignment tools (e.g., to identify CDR sequences as defined by Kabat, Chotia, and/or IMGT numbering, etc.). In some embodiments, the ABRs are identified computationally. In some embodiments, the ABRs are identified computationally using the Paratome web-based tool (Kunik, V. et al. (2012) Nucleic Acids Res. 40(Web Server issue):W521-4; Kunik et al. (2012) PLoS Comput. Biol. 8(2):e1002388). In some embodiments, the identified ABRs are used to predict the immunoglobulin's epitope on a polypeptide. In some embodiments, the epitope on the polypeptide is predicted computationally.

In some embodiments, the computational prediction relies on a machine-learning algorithm trained to recognize residue pairing preferences on a dataset of antibody/antigen complexes. In other embodiments the computational prediction is trained on sequences of pairs of experimentally verified epitopes-paratopes. In other embodiments the computational predictions is based on high throughput analysis of libraries and/or peptide derived from the antigen and their interaction with the antibodies and/or sera. Thus, the invention is also directed to a database on a computer readable medium comprising sequence of known ABRs and the structure of known antigenic polypeptides to which they bind. Although IgE antibodies are preferred, the antibody of the antibody/antigen complexes may be any antibody type. This includes IgG antibodies, T-cell receptors and all other types of antibodies. In preferred embodiments, the prediction tool is tested and optimized on data of interactions between IgE antibodies and their cognate allergens. In preferred embodiments, epitope prediction is not based on the 3D structure of the antigen or antigen-binding region, but only on the sequence of the ABRs.

In some embodiments, for identification of IgE binding regions, at least 5%, 10%, 15%, 20%, 25%, 30%, 35%, 40%, 45%, 50%, 55%, 60%, 65%, 70%, 75%, 80%, 85%, 90% or all of the known pairs of antibodies/antigen binding regions of the training set are IgE antibodies/antibody binding regions. For identifying the antigen-binding-regions of allergenic polypeptides for IgE antibodies, if antibodies other than IgE are used in the training set, the algorithm is optimized to succeed with IgE antibodies.

In other embodiments, for identification of T-cell receptor binding regions, the algorithm is trained to recognize HMC-peptide interaction for peptides derived from food proteins. Alternatively, or additionally, the algorithm is trained to predict TCR-MHC-peptide or TCR-peptide intereaction for peptides derived from food proteins.

In some embodiments, the epitope is predicted using the PEASE tool (Sela-Culang, J. et al. (2014) Structure 22(4):646-57; Sela-Culang et al. (2015) Bioinformatics 31(8):1313-5). In some embodiments, the predicted epitope is validated. In some embodiments, the predicted epitope is validated experimentally. Methods of experimentally testing antibody epitopes are known in the art, including, for example, by performing an antibody cross blocking assay, by performing an assay comprising screening a library, by performing mutation analysis, by deuterium exchange analysis, by peptide binding assays, and/or by x-ray crystallographic studies. In some embodiments, the epitope is validated by performing an antibody cross blocking assay and/or an assay comprising screening a library. In some embodiments, the validating uses libraries and/or peptides derived from the allergen to assess the importance of specific amino acids in specific positions for binding. In some embodiments, the library comprises a library of mutations to a subset or all of the amino acid residues of the allergenic polypeptide. In some embodiments, the library is a yeast display library. In some embodiments, the library is a phage display library.

### Allergenic Polypeptides

In some embodiments, the methods described herein are directed to identifying one or more epitopes on any allergenic polypeptide known in the art. Sources of polypeptides that are known or suspected to cause allergy or specific hypersensitivity may include, without limitation, plants, plant pollens, mold spores, animals, animal dander, house dust, foods, feathers, dyes, cosmetics, and any other source of allergenic polypeptides known in the art. Allergen-specific B cells may be cells that produce one or more immunoglobulins specific for any polypeptide from a source known or suspected to cause allergy or specific hypersensitivity.

In some embodiments, the allergenic polypeptide is a polypeptide naturally found in one or more non-food sources and/or products made or derived from the non-food source. In some embodiments, the allergenic polypeptide is a polypeptide naturally found in a non-food source that is known or suspected to induce allergy or specific hypersensitivity in one or more individuals. Examples of non-food sources known or suspected to induce allergy or specific hypersensitivity in one or more individuals may include, without limitation, animal products (e.g., Fel d 1, animal fur, animal dander, cockroach calyx, wool, dust mite excretion, etc.), medication (e.g., protein drugs, etc.), insect stings, mold spores, and plant pollens (e.g., ryegrass pollen, timothy-grass pollen, ragweed pollen, plantago pollen, nettle pollen, Artemisia vulgaris pollen, Chenopodium album pollen, sorrel pollen, birch pollen, alder pollen, hazel pollen, hornbeam pollen, Aesculus pollen, willow pollen, poplar pollen, Platanus pollen, Tilia pollen, Olea pollen, Ashe juniper pollen, Alstonia scholaris pollen, etc.).

In some embodiments, the allergenic polypeptide is a polypeptide naturally found in one or more food sources and/or products made or derived from the food source. In some embodiments, the allergenic polypeptide is a polypeptide naturally found in a food source that is known or suspected to induce allergy or specific hypersensitivity in one or more individuals. Examples of foods/food sources known or suspected to induce allergy or specific hypersensitivity in one or more individuals may include, without limitation, milk, eggs, fish (e.g., Alaskan Pollock, carp, cod, dogfish, mackerel, salmon, sole, tuna, etc.), crustacean shellfish (e.g., crab, lobster, shrimp, etc.), molluscan shellfish (e.g., abalone, horned turban, limpets, mussels, octopus, oysters, scallops, snails, squid, whelks, etc.), seeds such as nuts/tree nuts/(e.g., almonds, buckwheat, Brazil nuts, cashews, chestnuts, cocoa seeds, cotton seeds, flax seeds, hazelnuts, Macadamia nuts, mustard, pecans, pine nuts, poppy seeds, pumpkin seeds, sesame seeds, sunflower seeds, walnuts, etc.) or legumes (e.g., chickpeas, lentils, lupin, peanuts, peas, soybeans/soy, etc.). In other embodiments the food is cereals/grains (e.g., barley, corn/maize, oats, rice, rye, wheat, etc.), vegetables (e.g., celery/celeriac, asparagus, bell pepper, cabbage, carrot, lettuce, potato, turnip, zucchini, etc.), and fruit (e.g. apple, peach, kiwi, pumpkin, Acerola, apricot, avocado, banana, cherry, coconut, date, fig, grape, lychee, mango, melon, orange, papaya, passion fruit, pear, persimmon, pineapple, pomegranate, prune, strawberry, tomato, etc.). In some embodiments, the food source is one or more of peanuts, tree nuts, wheat, soy, milk, eggs, fish, shellfish, or any other allergen.

In some embodiments, the allergenic polypeptide is a polypeptide belonging to a protein family that is known or suspected to induce allergy or specific hypersensitivity in one or more individuals. Examples of protein families known or suspected to induce allergy or specific hypersensitivity in one or more individuals may include, without limitation, the prolamin superfamily, the cupin superfamily, the profilins, and Bet v-1-related proteins.

In some embodiments, the methods described herein are directed to identifying one or more epitopes on an allergenic polypeptide, and further, mutating one or more amino acid residues of the allergenic polypeptide. In some embodiments, the method comprises mutating one or more amino acid residues of the allergenic polypeptide in one or more of the identified epitopes. In some embodiments, the method comprises mutating 1, 2, 3, 4, 5, 6, 7, 8, 9, 10, 11, 12, 13, 14, 15, 16, 17, 18, 19, 20, 21, 22, 23, 24, or 25 or more amino acid residues of the polypeptide. In some embodiments, the one or more mutations destroy one or more (or all) of the identified epitopes on the polypeptide. Methods for making polypeptides comprising one or mutations are well known to one of ordinary skill in the art. In some embodiments, the one or more mutations are conservative mutations. In some embodiments, the one or more mutations are non-conservative mutations. In some embodiments, the one or more mutations are a mixture of conservative and non-conservative mutations.

The mutation of this aspect of the present invention may be a substitution, a deletion or an insertion.

According to a specific embodiment, the mutation does not affect the function of the allergenic polypeptide.

Methods of introducing nucleic acid alterations to a gene of interest are well known in the art [see for example Menke D. Genesis (2013) 51: 618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences. In some embodiments, the generation of the alterations in the sequences of the genes may be achieved by screening sequences of existing plants in search of an existing variant of the desired sequence. Then, this existing sequence will be introduced into the genome of the target genome by crossbreeding. In other embodiments the desired variations will be introduced by introducing random mutagenesis, followed by screening for variants where the desired mutations occurred, followed by crossbreeding.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8, 124,369; 8, 129,134; 8,133,697; 8,143,015; 8,143,016; 8, 148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA. Modified versions of the Cas9 enzyme containing two inactive catalytic domains (dead Cas9, or dCas9) have no nuclease activity while still able to bind to DNA based on gRNA specificity.

The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], To12 [Kawakami et al. PNAS (2000) 97 (21): 11403-11408] or Frog Prince [Miskey et al. Nucleic Acids Res. Dec. 1, 2003 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

In some embodiments, the one or more mutations do not disrupt the function of the polypeptide (e.g., do not disrupt the function of the mutated polypeptide relative to the function of the corresponding unmutated polypeptide). In some embodiments, the one or more mutations do not significantly affect the growth of the plant (for example production of seeds, number of seeds, size of seeds) from where the food is derived. In some embodiments, the one or more mutations do not disrupt native protein-protein interactions of the polypeptide (e.g., the mutated polypeptide retains the ability to form substantially the same protein-protein interactions as the corresponding unmutated polypeptide). In some embodiments, the one or more mutations do not disrupt the three-dimensional structure of the polypeptide (e.g., the mutated polypeptide retains substantially the same three-dimensional structure as the corresponding unmutated polypeptide). In some embodiments, the one or more mutations do not disrupt the folding of the polypeptide (e.g., the mutated polypeptide retains substantially the same protein folding as the corresponding unmutated polypeptide). In some embodiments, the one or more mutations do not disrupt the translation of the polypeptide (e.g., the mutated polypeptide is translated with the same timing, at the same rate, to the same levels, etc. as the corresponding unmutated polypeptide). In some embodiments, the one or more mutations do not disrupt the normal cellular localization of the polypeptide (e.g., the mutated polypeptide retains substantially the same cellular localization as the corresponding unmutated polypeptide). In some embodiments, the one or more mutations do not disrupt any post-translational modifications on the polypeptide (e.g., the mutated polypeptide retains substantially the same post-translational modification profile as the corresponding unmutated polypeptide).

Exposure of subjects to the modified protein allergens of the present invention may bring about tolerance in an allergic subject such that they become unresponsive or less sensitive to the protein allergen and do not develop severe symptoms of an immune response upon such exposure. In addition, administration of the modified protein allergen of the invention may modify the lymphokine secretion profile as compared with exposure to the naturally-occurring protein allergen or portion thereof (e.g. result in a decrease of IL-4 and/or an increase in IL-2). Furthermore, exposure to such a protein allergen may influence T-cell subpopulations which normally participate in the response to the allergen such that these T-cells are drawn away from the site(s) of normal exposure to the allergen (e.g. nasal mucosa, skin and lung) towards the site(s) of therapeutic administration of the fragment or protein allergen. This redistribution of T-cell sub-populations may ameliorate or reduce the ability of an individual's immune system to stimulate the usual immune response at the site of normal exposure to the allergen, resulting in a diminution in allergic symptoms. Moreover, exposure to the modified protein may change the balance of IgG antibodies against the allergen and IgE antibodies against it. This may result in amilioration or elimination of the allergic reaction upon exposure to the original allergen. Thus, the modified protein may be used as a treatment to eliminate sensitivities.

Reducing or Eliminating Binding and/or Reactivity

In some embodiments, the methods of the present disclosure are directed to assessing binding and/or reactivity of serum and/or one or more allergen-specific B cells and/or one or more antibodies (e.g., an IgE antibody) or T cell receptors to any of the allergenic polypeptides as described herein. In some embodiments, the methods of the present disclosure are directed to assessing binding and/or reactivity of serum and/or one or more allergen-specific B cells and/or one or more antibodies (e.g., an IgE antibody) and/or T cell receptors to polypeptides (e.g., mutated polypeptides) comprising one or more (e.g., one or more, two or more, three or more, four or more, five or more, six or more, seven or more, eight or more, nine or more, 10 or more, etc.) substitutions to the amino acid sequence of any of the allergenic polypeptides of the present disclosure. Methods of assessing binding of serum and/or allergen-specific B cells and/or antibodies and/or T cell receptors to a polypeptide are known in the art, and may include, without limitation, performing an ELISA assay, performing western blot analysis, performing a radioimmunoassay (RIA), performing Surface Plasmon Resonance (SPR), performing thermopheresis, performing a competition assay, and performing isothermal titration calorimetry. In some embodiments, the methods of the present disclosure comprise identifying mutated polypeptides comprising one or more substitutions to the amino acid sequence of any of the allergenic polypeptides of the present disclosure with reduced binding and/or reactivity of serum and/or one or more allergen-specific B cells and/or one or more antibodies and/or T cell receptors to the mutated polypeptides relative to the allergenic polypeptide lacking the one or more substitutions. In some embodiments, binding and/or reactivity to the mutated polypeptide comprising the one or more substitutions is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65, at least about 70%, at least about 75%, at least about 80%, at least about 85%, at least about 90%, at least about 95%, or at least about 99% relative to binding and/or reactivity to the allergenic polypeptide lacking the one or more substitutions. In some embodiments, binding and/or reactivity to the mutated polypeptide comprising the one or more substitutions is reduced by at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 100 fold, or at least about 1000 fold relative to binding and/or reactivity to the allergenic polypeptide lacking the one or more substitutions. In some embodiments, binding and/or reactivity of serum and/or one or more allergen-specific B cells and/or one or more antibodies (e.g. IgE antibodies), and/or T cell receptors to the mutated polypeptide is eliminated (e.g., binding and/or reactivity is undetectable, the serum/one or more allergen-specific B cells/antibodies, and/or T cell receptors no longer bind to the mutated polypeptide, etc.). In some embodiments, the antibody is an IgA, IgE, or IgG antibody. In some embodiments, the antibody is an IgE antibody.

In some embodiments, polypeptides (e.g., mutated polypeptides), or polynucleotides encoding the same, comprising one or more substitutions that reduce or eliminate binding and/or reactivity relative to the allergenic polypeptide are sequenced to identify their one or more substitutions. Methods of sequencing polynucleotides and/or polypeptides are known in the art.

In some embodiments, the identified one or more substitutions are the substitutions that reduce or eliminate binding and/or reactivity to the polypeptide (e.g., mutated polypeptide) relative to the allergenic polypeptide. In some embodiments, the identified one or more substitutions that reduce and/or eliminate binding and/or reactivity are substitutions in one or more allergenic epitopes on the allergenic polypeptide. In some embodiments, the polypeptides (e.g., mutated polypeptides) comprising the one or more substitutions that reduce and/or eliminate binding relative to the allergenic polypeptide are de-epitoped polypeptides.

### Reducing or Eliminating Allergenicity of the Allergenic Polypeptides

In some embodiments, the methods described herein are directed to identifying one or more epitopes on an allergenic polypeptide, and further, mutating one or more amino acid residues of the allergenic polypeptide to reduce or eliminate allergenicity of the polypeptide. In some embodiments, the one or more mutations reduce or eliminate allergenicity of the polypeptide, thereby de-epitoping the previously allergenic polypeptide. In some embodiments, the one or more mutations reduce or eliminate allergenicity of the polypeptide relative to a polypeptide lacking the one or more mutations.

In some embodiments, reducing or eliminating allergenicity of a polypeptide comprises making one or more mutations to the polypeptide to reduce or eliminate the affinity of an allergen-specific antibody for the polypeptide. In some embodiments, the affinity of the allergen-specific antibody for the mutant polypeptide is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65, at least about 70%, at least about 75%, at least about 80%, at least about 85%, at least about 90%, at least about 95%, or at least about 99% relative to the affinity of the allergen-specific antibody for a polypeptide lacking the one or more mutations. In some embodiments, the affinity of the allergen-specific antibody for the mutant polypeptide is reduced by at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 100 fold, or at least about 1000 fold relative to the affinity of the allergen-specific antibody for a polypeptide lacking the one or more mutations. In some embodiments, the affinity of the allergen-specific antibody for the mutant polypeptide is eliminated (e.g., binding of the allergen-specific antibody to the mutant polypeptide is undetectable, the allergen-specific antibody no longer binds to the mutant polypeptide, etc.).

Methods for measuring antibody affinity for a polypeptide are known in the art, and may include, without limitation, performing an ELISA assay, performing a radioimmunoassay (RIA), performing Surface Plasmon Resonance (SPR), performing thermopheresis, performing a competition assay, and performing isothermal titration calorimetry. In some embodiments, the allergen-specific antibody is an IgA, IgE, or IgG antibody. In some embodiments, the allergen-specific antibody is an IgE antibody.

In some embodiments, reducing or eliminating allergenicity of a polypeptide comprises making one or more mutations to the polypeptide to reduce or eliminate the reactivity of serum isolated from a subject to the mutated polypeptide. In some embodiments, the subject is allergic to the unmutated (e.g., wild-type) polypeptide. In some embodiments, the subject suffers from a non-food allergy and/or specific hypersensitivity. In some embodiments, the subject suffers from a non-food allergy and/or specific hypersensitivity to any one or more non-food sources as described herein. In some embodiments, the subject suffers from a food allergy and/or specific hypersensitivity. In some embodiments, the subject suffers from a food allergy and/or specific hypersensitivity to any one or more food sources as described herein. In some embodiments, the food allergy is an allergy to peanuts, tree nuts, wheat, soy milk, eggs, fish, shellfish, or any combinations thereof. In some embodiments, the serum is isolated from peripheral blood of the subject. In some embodiments, the reactivity of the serum to the mutant polypeptide is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65, at least about 70%, at least about 75%, at least about 80%, at least about 85%, at least about 90%, at least about 95%, or at least about 99% relative to reactivity of the serum to a polypeptide lacking the one or more mutations. In some embodiments, the reactivity of the serum to the mutant polypeptide is reduced by at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 100 fold, or at least about 1000 fold relative to reactivity of the serum to a polypeptide lacking the one or more mutations. In some embodiments, reactivity of the serum to the mutant polypeptide is eliminated. Methods of measuring serum reactivity to a polypeptide are known in the art, including, for example, by ELISA assay, by western blot analysis, etc.

In some embodiments, reducing or eliminating allergenicity of a polypeptide comprises making one or more mutations to the polypeptide to reduce or eliminate responsiveness to one or more tests of allergenicity in a subject. In some embodiments, the subject suffers from a non-food allergy and/or specific hypersensitivity to any one or more non-food sources as described herein. In some embodiments, the subject suffers from a food allergy and/or specific hypersensitivity. In some embodiments, the subject suffers from a food allergy. In some embodiments, the subject suffers from a food allergy to any one or more food sources as described herein. In some embodiments, the food allergy is an allergy to peanuts, tree nuts, wheat, soy milk, eggs, fish, shellfish, or any combinations thereof. In some embodiments, the subject is allergic to a polypeptide lacking the one or more mutations. Methods of testing allergenicity of a polypeptide are known in the art, and may include, without limitation, skin-prick tests, food challenge tests, and blood tests.

In some embodiments, reducing or eliminating allergenicity of a polypeptide comprises making one or more mutations to the polypeptide to reduce or eliminate one or more signs and/or symptoms of an allergic reaction in a subject exposed to the polypeptide. In some embodiments, reducing or eliminating allergenicity of a polypeptide comprises making one or more mutations to the polypeptide to reduce or eliminate one or more signs and/or symptoms of an allergic reaction in a subject exposed to the polypeptide relative to the one or more signs and/or symptoms of an allergic reaction in a subject exposed to a corresponding polypeptide lacking the one or more mutations. In some embodiments, the subject suffers from a non-food allergy and/or specific hypersensitivity to any one or more non-food sources as described herein. In some embodiments, the subject suffers from a food allergy and/or specific hypersensitivity. In some embodiments, the subject suffers from a food allergy. In some embodiments, the subject suffers from a food allergy to any one or more food sources as described herein. In some embodiments, the food allergy is an allergy to peanuts, tree nuts, wheat, soy milk, eggs, fish, shellfish, or any combinations thereof. In some embodiments, the subject is allergic to a polypeptide lacking the one or more mutations. Signs and symptoms of an allergic reaction may include, without limitation, developing a rash, developing hives, itching (e.g., of the mouth, lips, tongue, throat, eyes, skin, etc.), swelling (e.g., of the lips, tongue, eyelids, the whole face, etc.), difficulty swallowing, a runny or congested nose, a hoarse voice, difficulty breathing, wheezing, shortness of breath, repetitive cough, diarrhea, abdominal pain, stomach cramps, lightheadedness, fainting, nausea, vomiting, shock, circulatory collapse, a weak pulse, pale or blue coloring of the skin, an unspecified sense of discomfort, and anaphylaxis.

Without wishing to be bound by theory, the methods described herein provide a novel approach to identifying allergen-specific antibody epitopes on allergenic polypeptides, as well as a novel approach to treating allergenic polypeptides by mutagenesis to effectively de-epitope the previously allergenic polypeptide. Without wishing to be bound by theory, the methods described herein represent an entirely novel strategy for treating food allergies; a strategy that will provide a number of beneficial outcomes for allergic individuals, their families, and society at large: 1) providing foods and products that are safe to handle and consume for allergic individuals, 2) allowing allergic individuals to no longer avoid consuming otherwise healthy foods (thereby addressing the nutritional disadvantages of food avoidance), 3) reducing the risk of accidental exposure by increasing use of non-allergenic foods, 4) reducing the negative economic impacts of food allergies, 5) reducing/eliminating the need to perform potentially risky treatments on allergic individuals, 6) mapping epitopes for individual patients, thus offering the possibility of personalized tests that assess the risks of each person for various foods, 7) mapping epitopes for individual patients, thus offering the possibility of personalized nutrition recommendations, and 8) mapping epitopes for individual patients, thus offering the possibility of personalized treatment and exposure to environmental or medical reactants that are not necessarily food.

### EXAMPLES

The present disclosure will be more fully understood by reference to the following examples. They should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Predicting IgE Antibody Epitopes

The following results provided a proof of concept that: 1) by using antibody sequence data, it was possible to identify residues that were crucial for the recognition of an allergen by the immune system; and 2) these predictions were done computationally, with high precision, based solely on the antibody sequences and a model of the allergen structure alone. The surprising results described herein (i.e., the capability for highly accurate allergen-epitope prediction) opens up new possibilities for identifying and combatting food allergens.

### Methods

### Epitope Prediction

To computationally predict the epitope for the indicated IgE antibodies, the antibody binding regions (ABRs) of the antibodies were identified using the Paratome web-based tool, as described previously (Kunik, V. et al. (2012) Nucleic Acids Res. 40(Web Server issue):W521-4; Kunik et al. (2012) PLoS Comput. Biol. 8(2):e1002388). This web-based tool was designed to identify the boundaries of the ABRs on each of the variable loops of an antibody, and was shown to be superior over known methods for identifying CDRs. Using the Patatome tool, the paratope for each antibody was identified. Once the ABRs were identified, a machine learning based algorithm was used to predict the epitopes for each antibody. This algorithm was similar to the one, as described previously (Sela-Culang, J. et al. (2014) Structure 22(4):646-57; Sela-Culang et al. (2015) Bioinformatics 31(8):1313-5). However, it has been shown that allergy epitopes, which are typically recognized by IgE antibodies, (are different than other antibody epitopes, particularly those identified by IgG antibodies (see for example Pomés, A. (2010) Int Arch Allergy Immunol, 152, 1-11; Aalberse, R. C. and Crameri, R. (2011) Allergy, 66, 1261-1274; Dall'antonia, F., Pavkov-Keller, T., Zangger, K. and Keller, W. (2014) Methods, 66, 3-21. Thus, a prediction tool that was optimized to predict IgG epitopes was unlikely to preform well on IgE antibodies. In the new algorithm IgE epitopes were used to optimize parameters to achieve good performance of with high precision on IgE epitopes.

### Peptide Binding Analysis

Sera peptide binding was performed by fractionating samples and using electrotransfer to PVDF membranes (Millipore). After washing and blocking the membranes, the membranes were incubated overnight at 4° C. with sera (dilution 1:70), washed, and then incubated with alkaline-phosphatase-conjugated monoclonal anti-human IgE (Sigma; 1:500 dilution) at room temperature for 1.5 hours. The membranes were then developed by adding disodium 2-chloro-5-(4-methoxyspiro {1,2-dioxetane-3,2′-(5′-chloro) tricyclo[3.3.1.1] decan}-4-yl)-1-phenyl phosphate (Sigma).

### Results

### Predicting Epitopes on Two Allergens

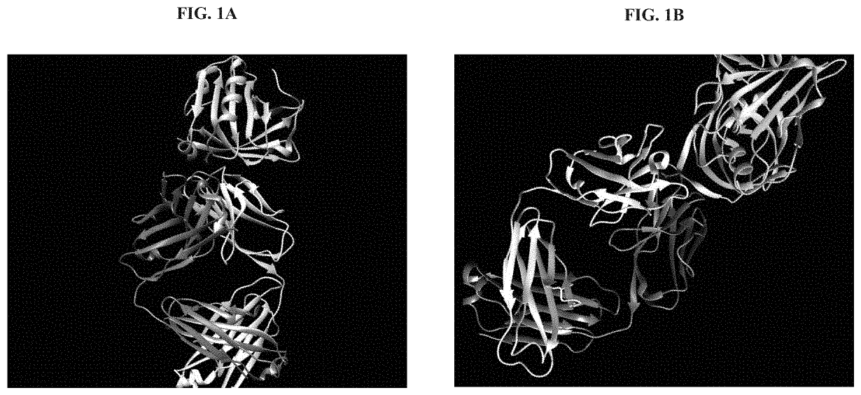

Two allergens that were solved in complex with a cognate IgE antibody were used as a model to test and validate the computational approach to epitope predictions. These two allergen-antibody complexes were 1) the complex of a beta-lactoglobulin allergen with an IgE antibody (PDB:2r56)(FIG. 1A), and 2) the cockroach allergen Bla g 2 complexed with an IgE antibody (PDB:2nr6)(FIG. 1B). Applying the epitope prediction scheme to these two complexes, key residues in the epitope of each allergen were predicted (the two strongest predicted residues are shown in red in FIGS. 1A-1B). These predicted residues included residue GLU127 in the beta-lactoglobulin allergen, and residue ASP68 in the cockroach allergen Bla g 2. The computationally identified residues were then checked against the experimentally identified epitope for each IgE antibody on the allergens. In the complex of a beta-lactoglobulin allergen with the IgE antibody, GLU127 of beta-lactoglobulin created several specific contacts with the antibody, including a salt bridge with the CDRH2 (FIG. 1A). In the complex of the cockroach allergen Bla g 2 complexed with the IgE antibody, ASP68A of Bla g 2 created multiple specific contacts with four different CDRs (FIG. 1B). Further analysis of the three top patches in each complex showed that two of the top predicted patches on each allergen overlapped with the observed epitope. In both cases, several of the top ten predictions were hotspots for the allergen-antibody complex. While these allergens were not food allergens, they demonstrated that, surprisingly, this computational approach worked for IgE epitope prediction on allergens.

Identifying Sera-Binding Epitopes on Polypeptides from Legumes

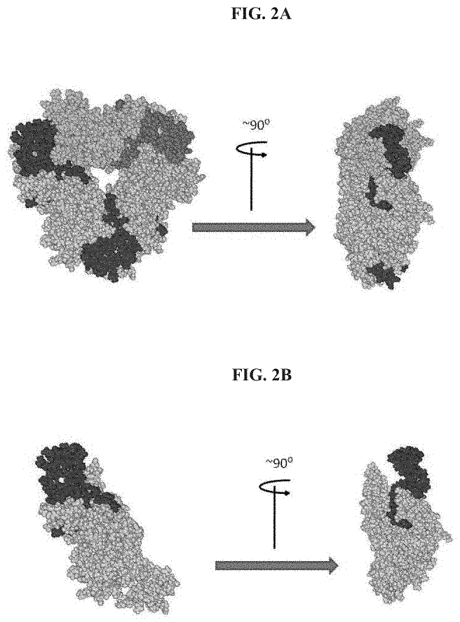

Epitopes for the chickpea provicilin precursor protein (a known food allergen) were predicted using the above described computational tools (FIGS. 2A-2B). Sera from chickpea-allergic patients were obtained to test whether they bind to peptides covering the sequence of the predicted epitope on the chickpea provicilin precursor protein. Some of these peptides were shown to bind to sera of chickpea-allergic individuals. These peptides were then used to further characterize the chickpea provicilin epitope.

The 3D structure of chickpea provicilin precursor was modeled using crystal structures of beta-conglycinin, a homologous vicilin from soybean seeds. As expected from the very high sequence identity (62%) and similarity (83%) between these two proteins, chickpea provicilin adopted the same cupin fold as beta-conglycinin (FIGS. 2A-2B). The structures of soybean beta-conglycinin used as templates for chickpea provicilin modeling were solved as homotrimers, in which the three cupin motifs were arranged around a three-fold symmetry axis (FIG. 2A). The monomers were held together by the interaction between a groove of one monomer and a complementary protrusion of the other one. While the stability of vicilin proteins to heat and digestion was well known, it was not clear whether IgE antibodies recognized provicilin as a trimer. The linear peptides identified in the sera binding analysis above suggested that this might be the case, as all of the peptides had at least a few residues exposed to the solvent in the trimeric form, which would not have necessarily been the case if the monomer form was the relevant form for IgE binding.

Two of the peptides that were shown to bind the sera shared a stretch of 10 residues, which suggested that these residues were likely part of a relevant portion of the protein necessary for antibody binding. Interestingly, all of the residues in this stretch that were exposed to the solvent in the monomer form (FIG. 2B) remained exposed in the trimer form as well (FIG. 2A). Of the other residues contained in the 10 residue stretch, only one remained exposed in the trimeric form (FIG. 2A). This further supported the relevance of the trimeric form of provicilin for IgE antibody binding. Most of the linear peptides identified were mapped to the groove region involved in the binding of another monomer (FIG. 2A). All identified linear peptides were mapped to two adjacent regions on the protein surface (FIG. 2A-2B). It was hard to tell whether all of the linear peptides identified were part of a single conformational epitope recognized by a single IgE antibody, or whether the two adjacent regions represented two partially overlapping epitopes corresponding to two different antibodies.

These results revealed that peptides covering predicted IgE epitopes on a given allergen were capable of being bound by sera from patients harboring an allergy to the allergen, while other peptides not identified by the computational epitope prediction analysis did not bind the sera. Taken together, these results demonstrated that IgE antibody epitopes could be predicted computationally, and further, that these epitopes could be verified using sera from allergic patients. Without wishing to be bound by theory, it is believed that these methods can be used to test and verify that an allergenic polypeptide has been effectively de-epitoped after mutagenesis by using patient sera according to the methodologies disclosed herein.

### Example 2: A Method for Identifying and De-Epitoping Allergens

### Producing Labelled Allergen

First, purified native allergen (either commercially available allergen, e.g., Ara h2, or recombinant purified allergen) is biotinylated at lysine residues using a water-soluble biotin-XX sulfosuccinimidyl ester according to the manufacturer's protocol (Invitrogen). Next, fluorescent multimers are formed by means of the stepwise addition of Alexa Fluor 488 streptavidin (Invitrogen) to the biotinylated allergen until a 1:4 molar ratio is reached, thereby fluorescently labelling the allergen.

Identifying Allergen-Positive Circulating B Cells from Patients

The identification of allergen-positive circulating B cells from patients is carried out as previously described (Patil, S. U. et al. (2015) J. Allergy Clin. Immunol. 136(1): 125-134). Briefly, peripheral blood mononuclear cells (PBMCs) are isolated by density gradient centrifugation (Ficoll-Paque Plus; GE Healthcare) from peripheral blood harvested from a subject. The PBMCs are stained with CD3-allophycocyanin (APC; clone OKT3; eBioscience), CD14-APC (clone 61D3, eBioscience), CD16-APC (clone CB16, eBioscience), CD19-APC-Cy7 (clone SJ25C1; BD Biosciences), CD27-phycoerythrin (clone M-T271; BD PharMingen), CD38-Violet 421 (clone HIT2, BD Biosciences), IgM-phycoerythrin-Cy5 (clone G20-127; BD PharMingen), and fluorescently-labelled allergen (e.g., Alexa Fluor 488-Ara h 2 multimer). Alexa Fluor 488⁺ B cells (allergen-positive B cells) are then identified and isolated from the rest of the stained PBMC population by flow cytometry using a FACS AriaIII instrument. Data is analyzed with FlowJo 8.8.7 software. Normalization of Alexa Fluor 488 is performed with Quantum Alexa Fluor 488 Molecules of Equivalent Soluble Fluorochrome (MESF) Beads (Bangs Laboratories). Single allergen-positive CD19⁺B cells, identified by FACS analysis, are sorted into individual wells of a 96 well plate (Eppendorf) containing 10 μL of first-strand buffer (Invitrogen) and 20 Units of recombinant RNasin Ribonuclease Inhibitor (Promega), and are frozen at −80° C.

### Single-Cell RT-PCR and Immunoglobulin Gene Amplification

Immunoglobulin genes are amplified from the frozen B cells using a nested RT-PCR protocol as follows: 3 μL of NP-40 and 150 ng of random hexamers are added to each well containing the frozen B cells, and the cells are lysed by heat treating the samples at 65° C. for 10 minutes, 25° C. for 3 minutes, and then a 4° C. incubation. The lysed cells are then subjected to a reverse transcription reaction using first-strand buffer (Invitrogen), 0.1 mM dithiothreitol (Invitrogen), 2.5 mM deoxynucletodie triphosphates (Invitrogen) and SuperScript III Reverse Transcriptase (Invitrogen) according to the manufacturer's protocol.

The resulting cDNA is then amplified by two rounds of nested PCR amplification. In the first round, 5 μL of template cDNA is amplified with Taq DNA polymerase (Invitrogen) according to the manufacturer's protocol. In the second round, 3 μL of the Taq-amplified template is amplified with Pfu DNA polymerase (Invitrogen) according to the manufacturer's protocol. A sample taken from the amplification products of the second PCR step is run on a 1.5% agarose gel to determine whether immunoglobulin heavy and light chains are successfully/adequately amplified. Successfully amplified products are then sequenced.

### Recombinant Antibody Production

Paired immunoglobulin heavy and light chains are selected after sequencing, and the DNA encoding the selected heavy and light chains (amplified in the second nested PCR step) is purified with the QIAquick 96 PCR Purification Kit (Qiagen). NEB® 5-alpha competent E. coli (New England Biolabs) are transformed with the heavy and light chain ligation products at 42° C. for 30 seconds, grown for 1.5 hours at 37° C., followed by selection on LB plates with 100 μg/mL ampicillin.

Ampicillin-resistant clones are selected and screened for vector insertion of the amplified immunoglobulin heavy and light chains. Vectors harboring the insertions are then amplified and purified from overnight liquid cultures (LB with 100 μg/mL ampicillin) of selected colonies using the QIAprep Spin Miniprep Kit (Qiagen). Plasmid DNA is then sequenced with colony PCR primers to determine similarity to previous sequences. Plasmid DNA (25 ng) from selected heavy and light chains are transfected into HEK293T cells using GenJet In Vitro DNA Transfection Reagent (SignaGen, Rockville, Md.) according to the manufacturer's protocol. Antibodies are purified from the transfected HEK293T cells according to standard techniques.

### Recombinant Antibody Characterization

Allergen specificity is then validated by ELISA assay (ImmunoCAP) for the recombinant antibodies purified from the transfected HEK293T cells according to the manufacturer's protocol.

### Epitope Prediction

To computationally predict the epitope for each recombinant antibody, first the antibody binding regions (ABRs) are identified using the Paratome web-based tool as described previously (Kunik, V. et al. (2012) Nucleic Acids Res. 40(Web Server issue):W521-4; Kunik et al. (2012) PLoS Comput. Biol. 8(2):e1002388). This web-based tool was designed to identify the boundaries of the ABRs on each of the variable loops of an antibody, and was shown to be superior over known methods for identifying CDRs. Using the Patatome tool, the paratope for each recombinant antibody is identified. Once the ABRs are identified, the PEASE tool is used to predict the epitopes for each recombinant antibody as described previously (Sela-Culang, J. et al. (2014) Structure 22(4):646-57; Sela-Culang et al. (2015) Bioinformatics 31(8):1313-5). Cross-blocking experiments using the purified antibodies is performed to filter and cluster predictions, as previously described (Sela-Culang, J. et al. (2014) Structure 22(4):646-57).

### Position Selection for Library Design

Based upon the predictions and the atlas of known epitopes, positions that are predicted to be crucial for antibody binding are selected. The selection is based on a combination score that takes into account: (i) score of the prediction; (ii) a multiple sequence alignment taking into consideration the conservation of the residues, where a higher score is given to less conserved residues; (iii) analysis of the effect of substitution of protein stability (e.g. by using MD and mutation analysis in DiscoveryStudio); and (iv) synergy with other putative substitutions within the same patch. The selected positions are altered, and amino acid variation is introduced at each position. The final mutational library also includes the wild-type residue at each selected position.

### Library Design

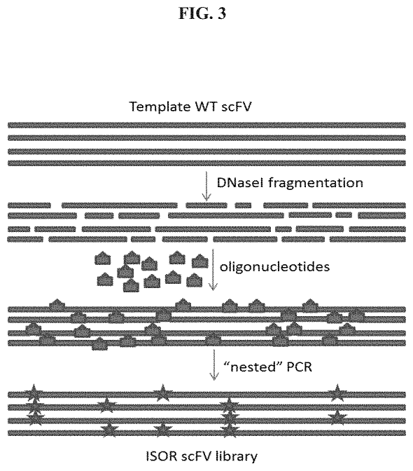

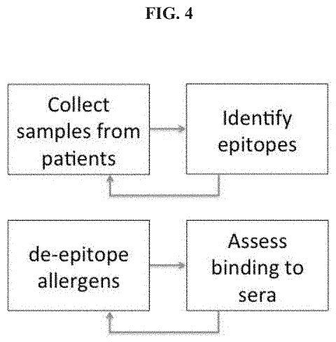

A library containing point mutations at each of the selected positions is generated by the dubbed incorporation synthetic oligos via gene reassembly method (ISOR) (See Herman, A. and Tawfik, D. S. (2007) Protein Eng. Des. Sel. 20(5):219-26). A template gene based on the allergen's wild-type sequence (“WT”) is ordered as a synthetic gene. Synthetic oligonucleotides containing the desired substitution at the selected position (complementary to the appropriate DNA region) are ordered at low purification grade. All substitutions in the library are encoded by a choice of codons that gives rise to the selected amino acids provided by the predictions, while minimizing the frequency of stop codons. A summary of this strategy is shown in FIG. 3. Briefly, template DNA is amplified using reverse and forward primers in order to obtain microgram amounts of template. The template DNA is fragmented with DNaseI, and 70-100 bp fragments are isolated. Next, the DNA fragments are mixed with various synthetic oligonucleotides containing the desired substitution at the selected position, and a PCR assembly reaction is performed (e.g., by using Pfu Turbo DNA polymerase (Stratagene)). The full length assembled genes are further amplified by “nested” PCR using appropriate forward and reverse primers containing a DNA sequence recognized by specific restriction enzymes. The DNA library harboring the desired diversity is then cloned into a pCTCON2 plasmid by ligating digested pCTCON2 with digested pure “nested” PCR products, and subsequently transforming electrocompetent E. coli cells with the purified ligation mix. Finally, the complexity of the library is assessed by sequencing random E. coli colonies. All plasmid-containing cells are pooled and the resulting EBY100 library is isolated and saved.

### Yeast Surface Display

Yeast surface display using the DNA library is performed as described previously (Chao, G. et al. (2006) Nat. Protoc. 1(2):755-68). Briefly, a yeast library is created having a diversity of about 1×10⁶ cells by transforming EBY100 yeast cells with the pCTCON2 plasmid library using an EZ transformation II kit (Zymo Research). The transformed cells are pooled, and the transformed yeast is grown in SDCAA media containing pen/strep overnight at 30° C. Next, 1×10¹⁰ transformed cells are collected by centrifugation, and supplemented with SGCAA medium, allowing for the surface expression of the mutated allergens expressed from the pCTCON2 plasmid. Protein induction is performed for 48 hours at 20° C. 1×10¹⁰ yeast cells expressing the mutated allergens are isolated, centrifuged, washed and incubated with biotinylated human antibodies against the allergen at desired concentration, as well as mouse a-Myc antibody (Santa Cruz, 1 μl/50 μl PBSF). The unbound antibodies are washed from the cells, and the cells are labeled for 30 minutes on ice with FITC-conjugated α-mouse IgG (Sigma, 1 μl/50 μl PBSF) to detect the expression level of the mutant allergens, and APC-conjugated streptavidin (Jackson, 0.5 μl/50 μl PBSF) to detect human antibodies bound to the mutant allergens. The cells are then washed, and subsequently analyzed and sorted by flow cytometry using a FACS AriaIII flow cytometer. Cells are sorted based on the expression level of the mutant allergens, and their ability to be bound by the allergen-specific human antibodies. Cells that express mutant allergens that are no longer bound by the allergen-specific antibodies (but still express the mutant allergen) are sorted and collected by gating on the APC-negative/FITC-positive cell population. The collected cells are grown in SDCAA medium with pen/strep for 24 hours, and glycerol stocks from this first round of mutant allergen selection are made. In parallel, plasmids are isolated and sequenced from these cells to identify point mutations in the allergens that allow for the robust expression of the protein, but that are no longer bound by the allergen-specific human antibodies. These allergens are effectively de-epitoped.

Without wishing to be bound by theory, it is believed that this procedure will identify correctly folded and well-expressed variants of allergenic polypeptides that are no longer recognized by allergen-specific antibodies. These variants of the allergenic polypeptides are good candidates for polypeptides that are structurally and functionally intact, yet are effectively de-epitoped. Without wishing to be bound by theory, this procedure embodies a novel approach to identifying allergen-specific antibody epitopes on allergenic polypeptides, and treating these polypeptides by mutagenesis to effectively de-epitope the previously allergenic polypeptide, representing an entirely novel strategy for treating food allergies. Without wishing to be bound by theory, this novel approach is believed to provide a number of beneficial outcomes for allergic individuals, their families, and society at large: 1) providing foods and products that are safe to handle and consume for allergic individuals, 2) allowing allergic individuals to no longer avoid consuming otherwise healthy foods (thereby addressing the nutritional disadvantages of food avoidance), 3) reducing the risk of accidental exposure by increasing use of non-allergenic foods, 4) reducing the negative economic impacts of food allergies, 5) reducing/eliminating the need to perform potentially risky treatments on allergic individuals, 6) mapping epitopes for individual patients, thus offering the possibility of personalized tests that assess the risks of each person for various foods, and 7) mapping epitopes that may cause undiagnosed sensitivities and reactions, thus identifying reactions to food or other reactants that affect the well-being of the patient.

1. A method for identifying an epitope on an allergenic polypeptide, the method comprising the steps of:
a) identifying antigen binding regions (ABRs) from the amino acid sequence of an antibody or T cell receptor which binds to the allergenic polypeptide; and
b) computationally predicting an epitope on the allergenic polypeptide for the identified ABRs, thereby identifying the epitope on the allergenic polypeptide.

2. A method for de-epitoping an allergenic polypeptide, the method comprising mutating one or more amino acid residues of an epitope on the allergenic polypeptide to generate one or more mutation in said identified epitope, thereby de-epitoping the allergenic polypeptide.

3. The method of claim 2, further comprising:
a) identifying antigen binding regions (ABRs) from the amino acid sequence of an antibody or T cell receptor which binds to the allergenic polypeptide; and subsequently
b) computationally predicting an epitope on the allergenic polypeptide for the identified ABRs; wherein steps a) and b) are performed prior to said mutating.

4. The method of claim 1, further comprising validating the computationally predicted epitope experimentally.

5. The method of claim 3, further comprising validating the computationally predicted epitope experimentally following step b) and prior to said mutating.

6. The method of claim 1, wherein said antibody or said T cell receptor is isolated from peripheral blood of an allergic subject.

7. The method of claim 1, wherein said antibody is isolated from serum of an allergic subject.

8. The method of claim 1, wherein said antibody or said T cell receptor is isolated from a subject that suffers from a food allergy.

9. The method of claim 8, wherein the food allergy is an allergy to a food selected from the group consisting of legumes, tree nuts, sesame, wheat, soy, milk, eggs, fish, seafood and rice.

10. The method of claim 8, wherein the food allergy is an allergy to a seed.

11. The method of claim 8, wherein the food allergy is a peanut allergy.

12. The method of claim 1, wherein the antibody is an IgA antibody, an IgE antibody, or an IgG antibody.

13. The method of claim 1, wherein the antibody is an IgE antibody.

14. The method of claim 1, wherein said ABRs are identified using the online tool Paratome.

15. The method of claim 1, wherein computationally predicting the epitope is performed using:
(i) a machine-learning algorithm trained to recognize residue pairing preferences on a dataset of antibody/antigen complexes;
(ii) a machine-learning algorithm trained to recognize whether a given antibody and a given antigen are likely to bind each other based on a data set of antibody-antigen interactions;
(iii) an algorithm trained to recognize HMC-peptide interaction for peptides derived from food proteins; or
(iv) an algorithm trained to predict TCR-MHC-peptide or TCR-peptide interaction for peptides derived from food proteins.

16-19. (canceled)

20. The method of claim 1, wherein validating the computationally predicted epitope experimentally comprises performing an antibody cross blocking assay or an assay comprising screening a library.

21. The method of claim 20, wherein the library is a yeast display library or a phage display library.

22. (canceled)

23. The method of claim 2, wherein said mutating comprises mutating at least two amino acid residues of the identified epitope.

24-25. (canceled)

26. The method of claim 2, wherein the mutation:
(i) reduces allergenicity of the allergenic polypeptide relative to an allergenic polypeptide lacking the mutation, wherein said allergenicity is measured by binding of serum, skin prick test or clinical assessment of patients after exposure to the polypeptide;
(ii) reduces the affinity of an antibody for the allergenic polypeptide relative to the affinity of the antibody to an allergenic polypeptide lacking the mutation, wherein said affinity is measured by a competition assay, ELISA, SPR or thermophoresis; and/or
(iii) reduces reactivity of serum isolated from the subject to the polypeptide relative to reactivity of the serum to a polypeptide lacking the mutation.

27-34. (canceled)

35. The method of claim 2, wherein the mutation does not:
(i) disrupt the function of the polypeptide;
(ii) disrupt the three-dimensional structure of the polypeptide and/or
(iii) disrupt folding of the polypeptide.

36-37. (canceled)

38. A method for de-epitoping an allergenic polypeptide, the method comprising the steps of:
a) constructing a library comprising mutated polypeptides or polynucleotides encoding the mutated allergenic polypeptides, wherein the mutated polypeptides comprise one or more modifications to the amino acid sequence of the allergenic polypeptide;
b) assessing binding of allergen-specific antibodies or allergen-specific T cell receptors to the mutated polypeptides; and
c) identifying mutated polypeptides with reduced binding to said allergen-specific antibodies or allergen-specific T cell receptors relative to the allergenic polypeptide.

39. The method of claim 38, wherein a function of the mutated polypeptides is not disrupted.

40. The method of claim 38, further comprising assessing the expression, folding and/or function of said mutated polypeptides.

41-42. (canceled)

43. The method of claim 38, wherein the allergen-specific antibodies or said allergen specific T cell receptors are isolated from a subject that suffers from a food allergy.

44. The method of claim 43, wherein the food allergy is an allergy to a food selected from the group consisting of legumes, tree nuts, sesame, wheat, soy, milk, eggs, fish, seafood and rice.

45. The method of claim 43, wherein the food allergy is a peanut allergy.

46. The method of claim 38, wherein the antibody is an IgA antibody, an IgE antibody, or an IgG antibody.

47. The method of claim 38, wherein the antibody is an IgE antibody.

48-49. (canceled)

50. The method claim 38, wherein the mutated polypeptides comprise at least two substitutions to the amino acid sequence of the allergenic polypeptide.

51. (canceled)

52. The method of claim 38, further comprising identifying one or more modifications in the mutated polypeptides that reduce allergenicity of the allergenic polypeptide.

53-98. (canceled)
